(19) United States
(12) Patent Application Publication
(10) Pub. No.: US 2010/0187324 A1
(43) Pub. Date: Jul. 29, 2010

(54) Method and System for Scent Delivery

(75) Inventors: Ilya Feygin, Mountainside, NJ (US); Helen Feygin, Mountainside, NJ (US); Lee Albert Castleman, Green Brook, NJ (US)

Correspondence Address: DEMONT & BREYER, LLC, 100 COMMONS WAY, Ste. 250, HOLMDEL, NJ, 07733, US

(73) Assignee: TECHELAN, LLC, Mountainside, NJ (US)

(21) Appl. No.: 12/687,678

(22) Filed: Jan. 14, 2010

Related U.S. Application Data
(60) Provisional application No. 61/144,622, filed on Jan. 14, 2009.

(51) Int. Cl. A61L 9/04; B01F 3/02

(52) U.S. Cl. 239/6; 239/34; 239/44; 239/419.5

(57) ABSTRACT

A scent-delivery system is disclosed that utilizes as least one of (1) a venturi-effect delivery device for promoting evaporation of volatile compounds or (2) a pendulum for energy-efficient movement of solid or liquid forms containing the volatile compounds. When the venturi-effect delivery device is used, a flow of air therethrough drives the evaporation of volatile compounds. The pendulum can be used to move the venturi-effect delivery device to generate the relative movement between the air and the device, hence creating a “flow” of air.

### STATEMENT OF RELATED CASES

This case claims priority of U.S. Provisional Patent Application 61/144,622 filed Jan. 14, 2009, and incorporates that Provisional application herein by reference.

### FIELD OF THE INVENTION

The present invention relates to dispensing devices for a volatile substance, such as a perfume, a deodorizing agent, etc.

### BACKGROUND OF THE INVENTION

Air fresheners are commonly used to mask bad odors and/or provide a desired scent or aroma in a home, office, or other environment. The scent permeates the environment as volatile compounds that compose the scent, etc., evaporate and diffuse throughout the air.

A variety of devices, both active and passive, are available for dispensing and dispersing air-freshening substances into the environment. Passive devices usually rely on the spontaneous evaporation of the volatile scent compounds at room temperature. Active devices typically adopt one of several approaches. In one approach, the scent is sprayed into the environment. In another approach, a heating element is used to heat a solid or liquid material to promote evaporation of the volatile scent compounds. In a third approach, a fan is used to accelerate the rate of evaporation and diffusion of scent compounds to the environment.

There are drawbacks to all of these conventional approaches to scent delivery. Passive devices typically provide a pronounced and immediate scent release, with the result that a majority of the scent is released in the first few days of use. Some active devices, such as spray devices, usually operate intermittently to preserve the fragrance material. As a consequence, the quantity of fragrance in the air is inconsistent, rising rapidly during a release and falling thereafter. Furthermore, a person exposed to such a pronounced fragrance release tends to become desensitized to the scent. That is, sensitivity (i.e., awareness) of the scent is retained to a relatively lower concentration if that concentration were to remain steady.

Air fresheners that rely on heating or forced air require a power source. Most heat-based devices utilize resistive heating wherein the device is electrically coupled to an electrical outlet. This limits placement options for the air freshener. Air fresheners that utilize a fan are necessarily more bulky than other types (to accommodate a motor, propeller and batteries) and, of course, they are noisier.

Accordingly, there is a need for a scent-emitting system for use in a home, workplace, or like environment that overcomes the aforementioned disadvantages and limitations of the aforementioned air fresheners.

### SUMMARY OF THE INVENTION

The present invention provides a system for the effective and prolonged evaporation/delivery of volatile substances capable of imparting perceptible and desirable benefits to the air into which they are diffused. For convenience, the illustrative embodiment of the present invention is referred to as a “scent-delivery system.” It is to be understood, however, that the system is suitable for delivering volatile compounds other than scents. For example, in various alternative embodiments, the scent-delivery system is used to deliver deodorizing agents, sanitizing agents, insect repellant, and the like, as well as various scents.

A scent-delivery system in accordance with some embodiments of the present invention utilizes (1) the venturi effect to facilitate scent delivery and (2) some type of mechanism to create a flow of air through a venturi-effect delivery device (to drive evaporation of volatile compounds). The well-known “venturi effect” is a reduction in fluid pressure that results when a fluid flows through a constricted section of pipe.

In some embodiments, the venturi-effect delivery device is itself moved to create a “flow” of air; in some other embodiments, air or gas is blown past or otherwise injected into the venturi-effect delivery device.

In some of the embodiments in which the venturi-effect delivery device is moved, a pendulum provides the motive force. In some other embodiments in which the venturi-effect delivery device is moved, other sources of energy and mechanisms are suitably used for that purpose.

A scent delivery system in accordance with some additional embodiments of the present invention utilizes the energy/motion of a pendulum to drive a source of a volatile compound (e.g., a vial containing perfume, a solid cake of deodorizer, etc.). Movement of the source increases the rate of evaporation of the volatile compound(s) contained therein. The pendulum is a particularly efficient way to move the source of the volatile compound(s). The high energy efficiency of the pendulum is due to the contribution of gravity to the pendulum's motion. In conjunction with the illustrative embodiment, the pendulum is multifunctional; it can drive a clock and/or serve a decorative purpose (i.e., wherein the clock's pendulum is simply “for show”). The “pendulum drive” can be used with or without the venturi-effect delivery device. That is, the pendulum can move a source of volatile compounds and promote the evaporation and dispersion thereof, even if not used in conjunction with the venturi-effect delivery device.

In one particularly preferred embodiment, a venturi-effect delivery device having an annular configuration is formed via a ring and a sphere. The sphere, which has a smaller diameter than the ring, is disposed within the ring. An annular gap—which defines the venturi—is formed between the surface of the sphere and the inside of the ring. In some embodiments, the ring is physically coupled to a reservoir, such as vial that contains a liquid, such as a perfume, etc. A wick is fitted against the inside surface of the ring and extends therefrom into the reservoir. Exposed to the liquid in the reservoir, the wick becomes saturated with the liquid. The venturi-effect delivery device is coupled to a pendulum by a fixture (e.g., hook, etc.) that depends from the top of the ring. With each swing of the pendulum, air moves through the aforementioned venturi-defining gap, generating a region of relatively lower pressure therein. The wick that is disposed against the inside of the ring is exposed to this region of low pressure. The low pressure hastens the evaporation of volatile compounds contained in the liquid that saturates the wick.

Some embodiments of the present invention provide an apparatus that comprises:

a pendulum; and

a venturi-effect delivery device for delivering a volatile compound to a region to be treated, wherein:
(i) the venturi-effect delivery system is coupled to the pendulum; and
(ii) the venturi-effect delivery system is oriented with respect to the pendulum so that when the pendulum swings, a venturi effect is generated by the venturi-effect delivery system.

Some further embodiments of the present invention provide an apparatus that comprises:

a pendulum; and

a source of a volatile compound, wherein:
(i) the source is coupled to the pendulum and moves with the pendulum; and
(ii) the source is in fluidic communication with the ambient environment, wherein movement of the pendulum causes relative motion between air and the source, the relative motion increasing the rate at which the volatile compound evaporates relative to the rate at which the volatile compound evaporates in the absence of the relative motion.

Some additional embodiments of the present invention provide a method for delivering a volatile compound into a region to be treated, wherein the method comprises:

moving a source of a volatile compound; and

generating a venturi effect via the movement, wherein the venturi effect results in an increase in a rate at which the volatile compound is delivered to the region relative to a rate of delivery in the absence of the venturi effect.

### BRIEF DESCRIPTION OF THE DRAWINGS

FIG. 1 depicts a scent-delivery system in accordance with the illustrative embodiment of the present invention.

FIG. 2 depicts an embodiment of a scent-delivery system of FIG. 1, wherein the venturi-effect delivery system is embodied as a venturi tube.

FIG. 3A depicts the scent-delivery system of FIG. 2, wherein a pendulum moves the venturi-effect delivery system, generating the movement of air needed to generate the venturi effect. FIG. 3A depicts the scent-delivery system swinging to the “left.”.

FIG. 3B depicts the scent-delivery system of FIG. 3A, but wherein the scent-delivery system swinging to the “right.”

FIG. 4 depicts the scent-delivery system of FIG. 2, wherein the venturi-effect delivery system remains stationary and a fan generates the movement of air needed to generate the venturi effect.

FIGS. 5A, 5B, and 5C. depict an alternative embodiment of a scent-delivery system, wherein the venturi-effect delivery system comprises a ring and sphere.

FIG. 6 depicts a further alternative embodiment of a scent-delivery system, wherein the venturi-effect delivery system comprises a ring and a sphere, and wherein the sphere also serves as a source for a scent or other volatile compound.

FIG. 7 depicts an embodiment of a scent-delivery system being used in conjunction with a clock.

FIG. 8 depicts a method for delivering a volatile compound in accordance with the illustrative embodiment of the present invention.

### DETAILED DESCRIPTION

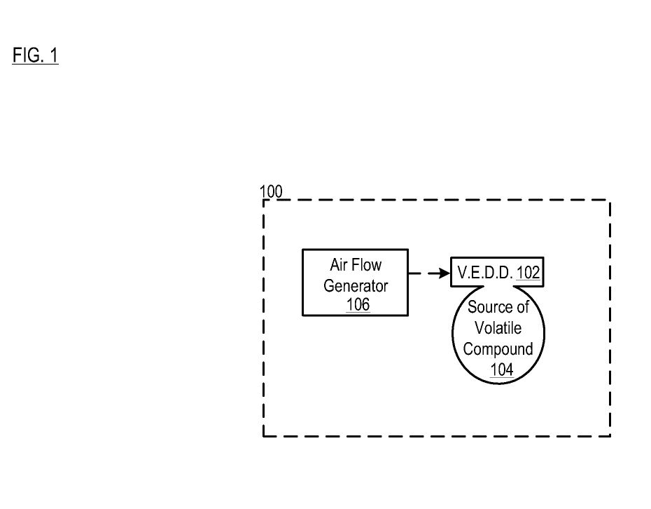

FIG. 1 depicts an embodiment of scent-delivery system 100 in accordance with the present invention. The salient features of scent-delivery system 100 include venturi-effect delivery device 102, source 104 of volatile compound, and air flow generator 106.

Venturi-effect delivery device 102 is a device, arrangement, etc., that utilizes the venturi-effect to promote the delivery of volatile compound from source 104 thereof. Various embodiments of the venturi-effect delivery device are depicted in the Figures and described later in this specification.

The venturi-effect delivery device is in fluidic communication with source 104 of volatile compound. Source 104 functions as a reservoir for the volatile compound that is to be delivered and dispersed. The volatile compound is typically associated with a carrier material, wherein the volatile compound is interspersed therein. The carrier can be in the form of a liquid, gel, or solid. In operation, the volatile compound diffuses out of the carrier material and, ultimately, into region to be treated. Venturi-effect delivery device promotes diffusion of the volatile compound out of the carrier and dispersion into the atmosphere, etc.

The operation of venturi-effect delivery device 102 requires a relative movement of air/gas with respect thereto, which is provided by air-flow generator 106. At this level of specificity, the air-flow generator is a “logical” concept. That is, in some embodiments, the venturi-effect delivery device is moved to generate a (relative) flow of air. In some other embodiments, the venturi-effect delivery device remains motionless while air/gas, etc. is forced into it. In still further embodiments, air/gas, etc., is forced past the outlet of venturi-effect delivery device 102 to create a venturi effect therein.

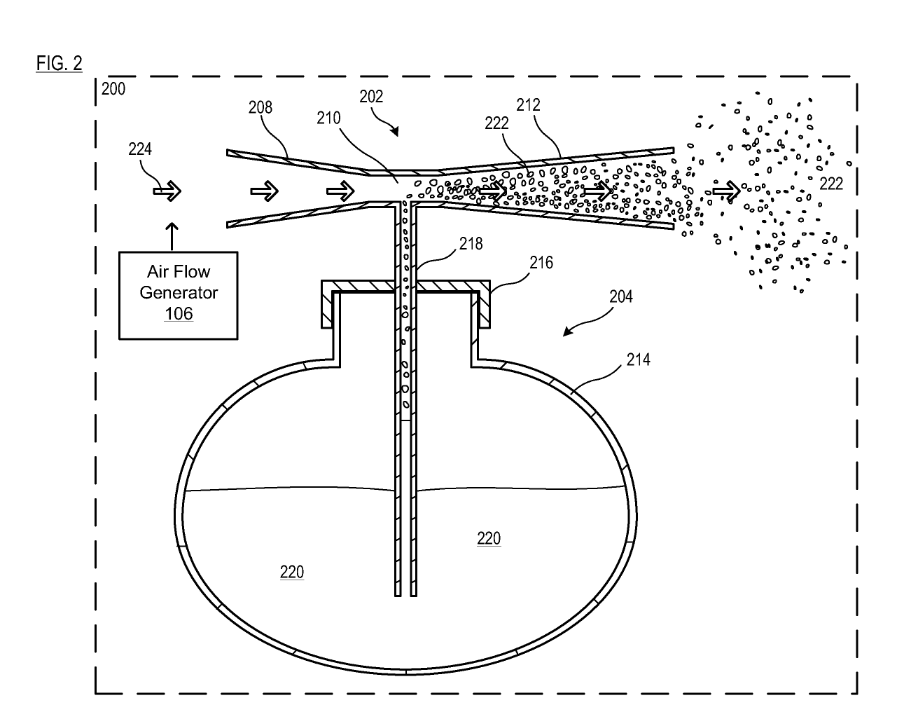

FIG. 2 depicts scent-delivery system 200, which provides a specific embodiment of venturi-effect delivery device 102 and source 104. In the embodiment depicted in FIG. 1, source 104 comprises vial 214 containing volatile compound/carrier 220. Vial 214 is sealed via cap 218.

Venturi-effect delivery device 102 is embodied as venturi tube 202. The arrangement and design of venturi tubes are well known in the art. Briefly, venturi tube 202 includes intake taper 208, throat 210, and exit taper 212.

Capillary 218 depends from throat 210 of venturi tube 202. Capillary tube 218 extends through cap 218 into volatile compound/carrier 220 within vial 214. In this fashion, capillary 218 places the venturi-effect delivery device 102 and source 104 in fluidic communication.

In operation, a flow of air/gas 224 is established through venturi tube 202 via air-flow generator 106 This creates reduced pressure in throat 210, which results in a suction effect that draws volatile compound/carrier 220 up capillary 218. The reduced pressure hastens evaporation of the volatile compound(s) from the carrier. Evaporated volatile compound 222 is drawn into throat 210 of venturi tube 202 and delivered to the ambient environment, etc. from exit taper 212.

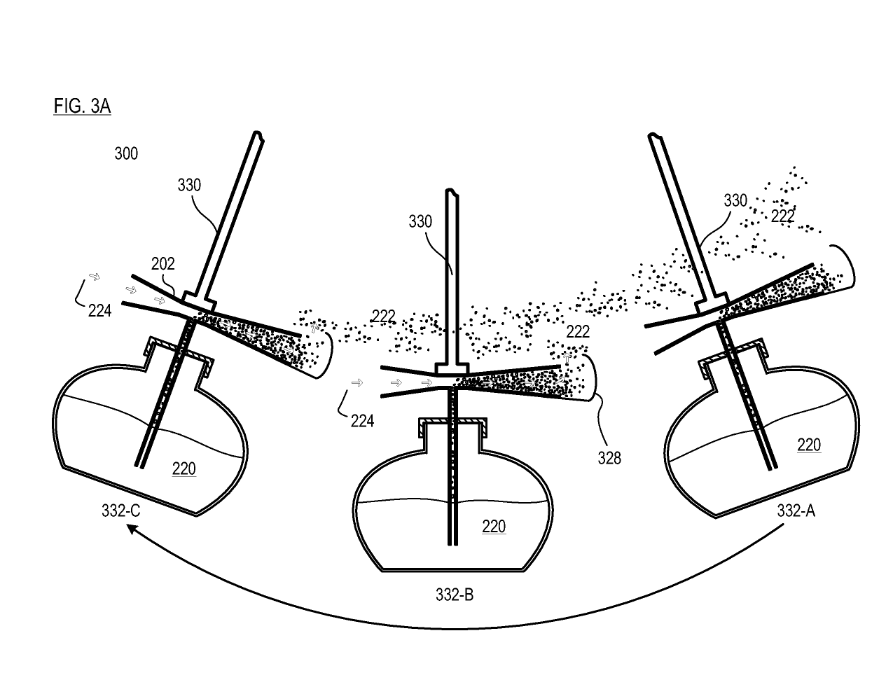

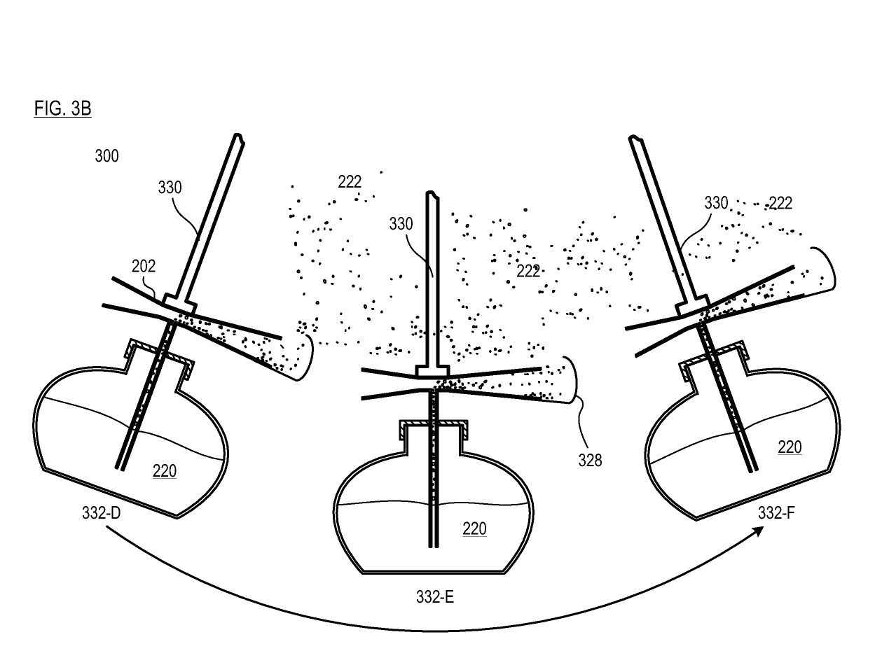

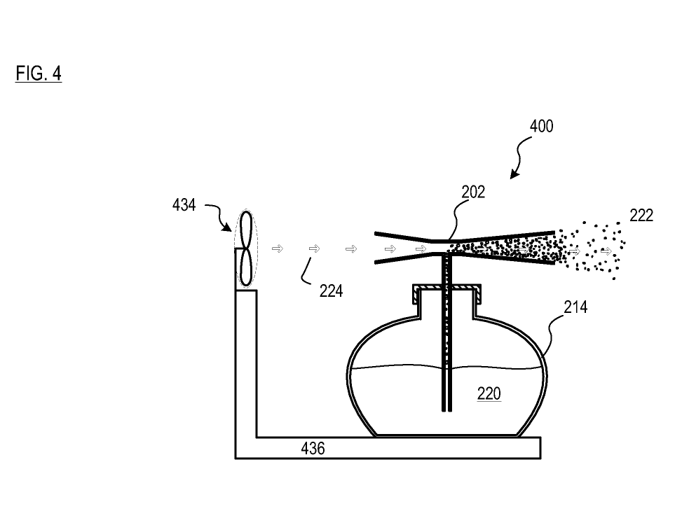

FIGS. 3A/3B depict an embodiment wherein air-flow generator 106 is a device that moves venturi-effect delivery device 102 and FIG. 4 depicts an embodiment wherein air-flow generator 106 is a device that moves air rather than the venturi-effect delivery device.

Referring now to FIGS. 3A and 3B, scent-delivery system 300 comprises venturi-effect delivery device 102 embodied as venturi tube 202, source 104 embodied as vial 214 containing volatile compound/carrier 220, and air-flow generator 106 embodied as an electrically-boosted pendulum. The pendulum is illustrated by arm 330. The arm is of course attached to a pivot point (not depicted) and is driven by some mechanism. The liquid-containing vial 214 and venturi tube 202 serve as the “bob” of the pendulum. Deflector 328 is attached to the exit taper of venturi tube 202.

FIG. 3A depicts scent-delivery system 300 in motion to the “left,” showing three “snap shots” 332-A, 332-B, and 332-C of the system, which represents the position of the system at various times through the pendulum's oscillation. Position 332-B is the equilibrium position of the pendulum, although in this illustration, the pendulum is not quiescent at the equilibrium position. Position 332-A depicts the system at the end of its oscillation toward the “right” and position 332-C depicts the system approaching the end of its oscillation toward the “left.”

As scent-delivery system 300 begins its movement toward the “left,” away from position 332-A, air 224 flows through venturi tube 202. The flow of air through the venturi tube continues until the system reaches the end of the pendulum's travel toward the left (just beyond position 332-C). The flow of air creates negative pressure in the throat of the venturi tube, thereby hastening evaporation of volatile compounds 222.

FIG. 3B depicts scent-delivery system 300 in motion to the “right,” showing three “snap shots” 332-D, 332-E, and 332-F of the system, which represents the position of the system at various times through the pendulum's oscillation. Position 332-E is the equilibrium position of the pendulum, although in this illustration, the pendulum is not quiescent at the equilibrium position. Position 332-D depicts the system at the end of its oscillation toward the “left” and position 332-F depicts the system approaching the end of its oscillation toward the “right.”

As scent-delivery system 300 begins its movement toward the “right,” away from position 332-D, there is minimal air flow through venturi tube 202 due to the presence of deflector 328. The deflector, however, assists in dispersing the previously released volatile compounds 222 as it “pushes” through the air.

In other embodiments, deflector 328 is not used, such that air will pass through venturi tube 202 regardless of its direction of swing.

Referring now to FIG. 4, scent-delivery system 400 comprises venturi-effect delivery device 102 embodied as venturi tube 202, source 104 embodied as vial 214 containing volatile compound/carrier 220, and air-flow generator 106 embodied as fan 434. In the illustrative embodiment, the fan depends from base 436, which houses a power source (e.g., batteries, etc.) for driving the fan. Fan 434 generates a flow 224 of air through venturi tube 202. The flow of air creates negative pressure in the throat of venturi tube 202, thus speeding evaporation of volatile compounds 222.

Fan 434 is a less efficient way to generate the air flow required for the venturi-effect delivery device than a pendulum. Even so, the presence of the venturi-effect delivery device improves the energy efficiency of scent delivery relative to a system that does not include such a device (and that does not use a pendulum).

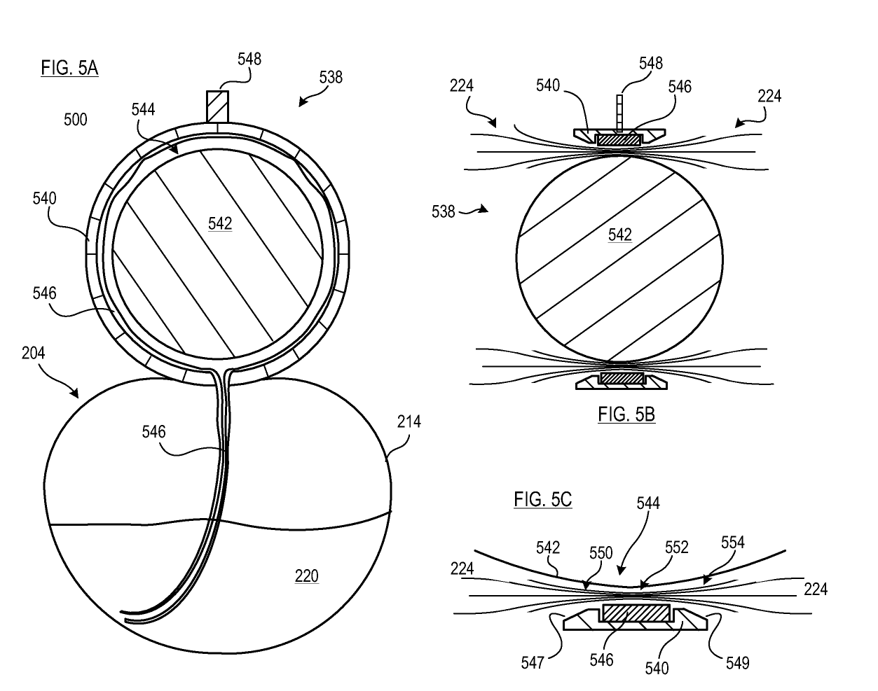

FIGS. 5A-5C depict scent-delivery system 500 (air-flow generator 106 not depicted). Scent-delivery system 500 includes annular venturi device 538, which is an alternative implementation of venturi-effect delivery device 102.

FIG. 5A, which is front sectional view of system 500, depicts annular venturi device 538 disposed on vial 214 containing volatile compound/carrier 220. In this embodiment, the annular venturi device comprises ring 540 and sphere 542. The diameter of the sphere is less than the inner diameter of ring 540. The sphere is disposed within the ring in concentric fashion. Gap 544 is defined between the surface sphere 542 and the inner surface of ring 540.

Wick 546 abuts the inner surface of ring 540 and extends into the liquid within vial 214. In some embodiments, wick 546 is fitted within a channel in ring 540 (see, e.g., FIG. 5B). Gap 544 is maintained in the presence of wick 546; that is, the wick does not fill the gap between the ring and sphere.

Hook 548 depends from the top of ring 540 and is used to couple annular venturi device 538 to, for example, a pendulum.

FIG. 5B depicts a side sectional view of annular venturi device 538. It can be seen that gap 544 between sphere 542 and wick 546/ring 540 defines a venturi. Referring also to FIG. 5C, which provides a close-up of gap 544, throat 552 is formed between sphere 542 and wick 546. Intake or exit taper 550 is formed between inclined surface 547 of ring 540 and the “upwardly” curving surface of sphere 542. And exit or intake taper 554 is formed between inclined surface 549 and the “upwardly” curving surface of sphere 542.

The operation of annular venturi device 538, at least in the embodiment depicted in FIGS. 5A-5C, is bi-directional. That is, the venturi-effect is generated regardless of the direction of air flow. It is for that reason that both “ends” of the “venturi” are designated as being inlet/exit tapers. Due to the fact the venturi is formed completely around the circumference of sphere 542, a large surface area is available for evaporation of the volatile compound(s).

In some other embodiments (not depicted), an emanator, rather than a wick 546, is fitted against the inner surface of ring 540. The emanator has a very large surface area and is especially adapted to facilitate the evaporation of volatile compounds. In such embodiments, a wick leads from vial 214 to the base of ring 540, at which point the wick fluidically couples to the emanator. The wick draws liquid from vial 214 via capillary action and delivers it to the emanator. Wicks for use with emanators can comprise a bundle of loosely-twisted fibers, braided or woven cord, tape or tube, non-woven material and porous polymer materials, such as sintered plastics.

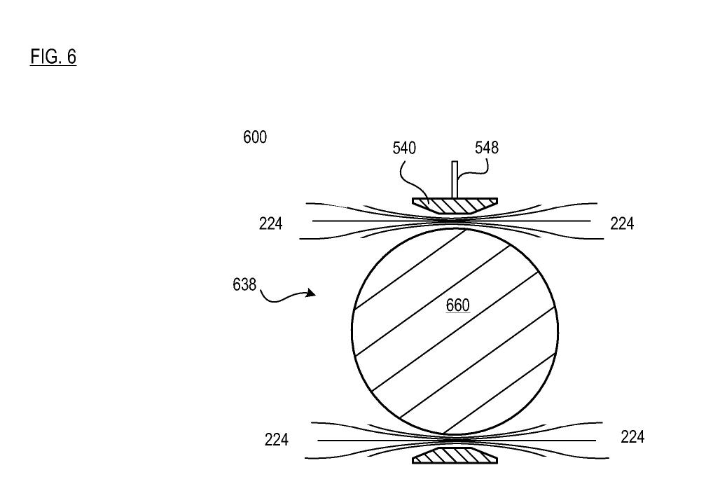

FIG. 6 depicts a side sectional view of scent-delivery system 600 (air-flow generator 106 for generating flow 224 of air is not depicted). In this embodiment, venturi-effect delivery device 104 has effectively the same structure of shown in FIGS. 5A-5C; that is, it is an annular venturi device that utilizes a sphere and ring 540. Unlike annular venturi device 538, however, annular venturi device 638 itself incorporates source 104 of volatile compound (rather than fluidically coupling to it, as the previous embodiments). This is accomplished by embodying source 104 as a solid form of the volatile compound/carrier, wherein the solid form is fashioned into spherical shape 660. In other words, the sphere has a dual function; its contour forms a portion of the venturi and its composition provides the volatile compound. Since the volatile compound is present in sphere 660, a wick, as used in the embodiment depicted in FIGS. 5A-5C, is not required. Movement of annular venturi device 538, or the introduction of air/gas into it while the device remains motionless, creates the venturi effect. This generates a low pressure region between the ring and the sphere to promote evaporation of the volatile compound(s) from sphere 660.

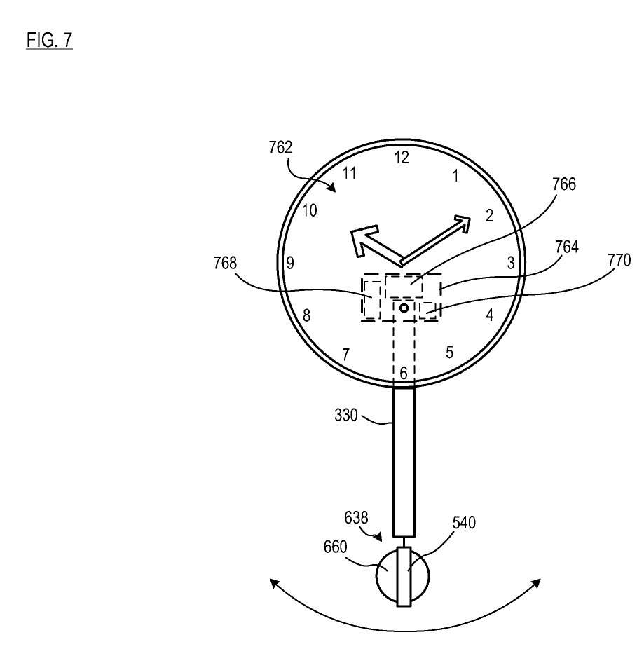

FIG. 7 depicts an embodiment of scent-delivery system 600 incorporating annular venturi device 638 and wherein air-flow generator 106 comprises an electrically-boosted pendulum, and wherein the pendulum is associated with clock 762. The pendulum is not used to keep time; rather it is simply a decorative accessory of the clock, while serving the dual purpose of being the air-flow generator for the annular venturi device.

The pendulum comprises control electronics 764 and arm 330. Annular venturi device 638 including sphere 660 and ring 540 serves as the “bob” of the pendulum. The pendulum is supported at a pivot point and is driven by drive system 766, which is part of control electronics 764. The drive system is powered by energy source 768, which in the illustrative embodiment is a battery. Control electronics 764 further includes controller 770, which is capable of varying certain aspects of the pendulum's movement. For example, in various embodiments, controller 770 controls one or more of amplitude, frequency, or angle of the pendulum. Changes in such parameters changes the rate of evaporation of the volatile compound(s) and, hence, the rate of delivery of fragrance, etc., to the space.

Movement of pendulum arm 330 in either direction creates a relative flow of air between sphere 660 and ring 540, thereby creating the venturi effect. As previously discussed, this creates a region of low pressure that promotes and enhances the evaporation of volatile compounds from sphere 660.

The size of venturi's throat will, of course, affect its performance. This is true for a “classic” venturi tube or the annular venturi device disclosed herein. Those skilled in the art will be able to readily determine the size of the gap to provide a desired level of performance. Typically, the gap will be less than about 1 millimeter. Of course, the gap size will be a function of the rate of air flow through the throat and other factors.

The use of a venturi-effect delivery device in conjunction with the pendulum is preferred, but the system is beneficial even in its absence. In particular, as previously mentioned, the pendulum is a very energy efficient way to create motion. The movement of air over a cake, gel, or vial of liquid comprising volatile compounds will increase the rate of evaporation of the volatiles relative to a situation in which air is still. As such, in some embodiments, a cake (spherical or otherwise), gel, or vial of liquid comprising volatile compounds is attached to the end of a pendulum arm.

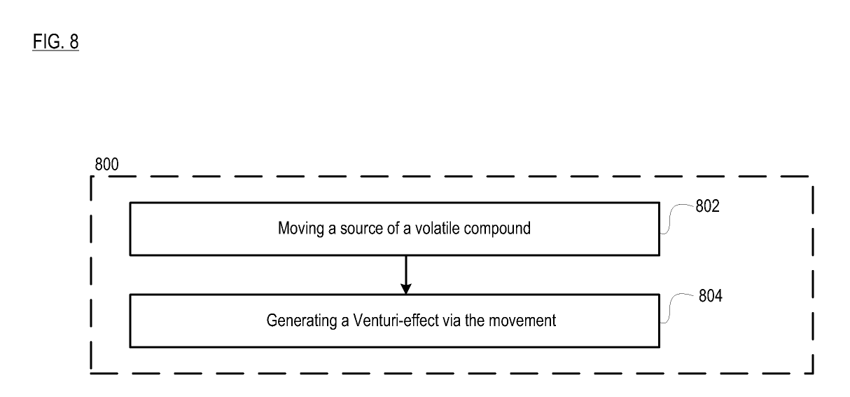

FIG. 8 depicts a method for delivering a volatile compound to a region to be treated, in accordance with the illustrative embodiment of the invention. In accordance with method 800, operation 802 recites moving a source of a volatile compound. As previously discussed, the movement can be generated, for example, by attaching a vial containing the compound/carrier, or attaching a solid form of the compound/carrier, to a pendulum.

Operation 804 recites generating a venturi-effect via the movement. As discussed above, physically coupling a venturi-effect delivery device to a pendulum creates a relative flow of air through the device. This results in a reduced pressure in the neck of the venturi, which results in an increase in the rate at which the volatile compound evaporates to the environment.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. An apparatus comprising:
a pendulum; and
a venturi-effect delivery system for delivering a volatile compound to a region to be treated, wherein:
(iii) the venturi-effect delivery system is coupled to the pendulum; and
(iv) the venturi-effect delivery system is oriented with respect to the pendulum so that when the pendulum swings, a venturi effect is generated by the venturi-effect delivery system.

2. The apparatus of claim 1 further comprising a source of a volatile compound, wherein the source of the volatile compound and the venturi-effect delivery system are in fluidic communication with each other.

3. The apparatus of claim 1 wherein the venturi-effect delivery system comprises a venturi tube.

4. The apparatus of claim 1 wherein the venturi-effect delivery system comprises:
a ring having a first diameter; and
a sphere having a second diameter that is smaller than the first diameter, wherein the sphere is disposed within the ring.

5. The apparatus of claim 4 wherein the sphere is the source of the volatile compound.

6. The apparatus of claim 4 wherein the venturi-effect delivery system further comprises a wick, wherein the wick is disposed against an inner surface of the ring, wherein a gap remains between the wick and the sphere, wherein the gap is suitably dimensioned and configured to function as a venturi.

7. The apparatus of claim 6 wherein the volatile compound is contained in a liquid carrier and is disposed in a reservoir, and further wherein the wick extends into the reservoir.

8. The apparatus of claim 1 wherein the pendulum is associated with a clock.

9. The apparatus of claim 2 wherein the volatile compound is selected from the group consisting of a fragrance, a deodorizing agent, a sanitizing agent, and an insect repellant.

10. The apparatus of claim 1 wherein aspects of the pendulum's motion are controllable for controlling the rate at which the volatile compound is delivered to the area to be treated.

11. An apparatus comprising:
a pendulum; and
a source of a volatile compound, wherein:
(iii) the source is coupled to the pendulum and moves with the pendulum; and
(iv) the source is in fluidic communication with the ambient environment, wherein movement of the pendulum causes relative motion between air and the source, the relative motion increasing the rate at which the volatile compound evaporates relative to the rate at which the volatile compound evaporates in the absence of the relative motion.

12. The apparatus of claim 11 wherein the source of the volatile compound is a solid.

13. The apparatus of claim 11 wherein the source of the volatile compound is a liquid.

14. The apparatus of claim 11 wherein the source of the volatile compound is fluidically coupled to a venturi-effect delivery system for delivering the volatile compound to the ambient environment.

15. The apparatus of claim 11 wherein aspects of the pendulum's motion are varied to control the rate at which the volatile compound evaporates.

16. A method for delivering a volatile compound to a region to be treated, wherein the method comprises:
moving a source of a volatile compound; and
generating a venturi effect via the movement, wherein the venturi effect results in an increase in a rate at which the volatile compound is delivered to the region relative to a rate of delivery in the absence of the venturi effect.

17. The method of claim 16 wherein the operation of moving a source further comprises moving the source via pendulum motion.

18. The method of claim 17 further comprising varying aspects of the pendulum motion to vary the rate at which the volatile compound is delivered to the region to be treated.

19. The method of claim 16 wherein the operation of generating a venturi effect further comprises generating the venturi effect via through an annular region.

20. The method of claim 16 wherein the volatile compound is selected from the group consisting of a fragrance, a deodorizing agent, a sanitizing agent, and an insect repellant.
